(19) United States
(12) Patent Application Publication
(10) Pub. No.: US 2019/0119711 A1
(43) Pub. Date: Apr. 25, 2019

(54) METHOD OF AND SYSTEM FOR PRODUCING A SYRUP WITH THE HIGHEST CONCENTRATION USING A DRY MILL PROCESS

(71) Applicant: Lee Tech LLC, San Jose, CA (US)

(72) Inventor: Chie Ying Lee, San Jose, CA (US)

(21) Appl. No.: 16/163,345

(22) Filed: Oct. 17, 2018

Related U.S. Application Data
(60) Provisional application No. 62/575,212, filed on Oct. 20, 2017.

(51) Int. Cl. C12P 19/02; C12P 7/06; C13K 1/02; A23K 10/38; B01D 21/26; B01D 3/00

(52) U.S. Cl. CPC C12P 19/02; C12P 7/06; B01D 3/001; A23K 10/38; B01D 21/262; C13K 1/02

(57) ABSTRACT

Method of and system for producing the highest concentration of a syrup (e.g., 80% dry matter) using a dry milling process are provided, which include (a) using backend grinding steps and devices for grinding thin stillage, (b) using a clean thin stillage system with two-disc centrifuges and two protein decanters, (c) adding an enriched syrup back to the syrup or evaporator, and (d) splitting two dryers into two independent parallel dryers using one dryer as a protein dryer and the other as a DDG dryer for producing DDGS.

### CROSS-REFERENCE TO RELATED APPLICATIONS:

This application claims priority from U.S. Provisional Patent Application Ser. No. 62/575,212, filed Oct. 20, 2017 and entitled “Method of and System for Producing Highest Syrup Concentration Using a Dry Mill Process,” which is incorporated herein by reference in its entirety for all purposes.

### FIELD OF THE INVENTION

The present invention relates to methods of and devices for dry milling systems and plants for alcohol production. More specifically, the present invention relates to methods of and systems for producing materials (e.g., nutrients) with a highly concentrated syrup while still achieving a high alcohol yield and increasing the co-product value containing the materials (e.g., oil, and animal feeds, including a high protein feed and low protein feed) in dry grinding ethanol plants.

### BACKGROUND OF THE INVENTION

FIG. 1 is a typical dry milling process 100 with a backend oil recovery system. FIG. 2 is a typical dry mill process 200 with a front-end grinding mill. FIG. 3 is a typical dry mill process 300 with a backend oil and protein recovery system.

The conventional methods of producing various types of alcohols from grains generally follow similar procedures depending on whether the process is operated wet or dry. Wet milling corn processing plants convert corn grains into several different co-products, such as germ (for oil extraction), gluten feed (high fiber animal feed), gluten meal (high protein animal feed), and starch-based products (such as ethanol, high fructose corn syrup, or food and industrial starch). Dry grinding or dry milling ethanol plants convert corns into two products, namely ethanol and distiller's grains with soluble. If the distiller's grains with soluble are sold as a wet animal feed, it is referred as Distiller's Wet Grains with Soluble (DWGS). If the distiller's grains with soluble is dried for making an animal feed, it is referred to as Distiller's Dried Grains with soluble (DDGS). In a standard dry grinding ethanol process, one bushel of corn yields approximately 8.2 kg (approximately 17 lbs.) of DDGS in addition to the approximately 10.3 liters (approximately 2.75 gal) of ethanol.

These co-products provide a critical secondary revenue stream that offsets a portion of the overall ethanol production costs. DDGS is generally sold as a low value animal feed, even though the DDGS contains 11% of oil and 33% of protein. Some plants have started to modify the typical process to separate the valuable oil and protein from DDGS.

Currently, there are over 120 plants with the backend oil recovery system as shown in FIG. 1, and four plants with protein recovery systems using a process as shown in FIG. 3. These processes are disclosed in the U.S. Patent Application Publication No. 2014/0317945; titled “METHODS FOR PRODUCING A HIGH PROTEIN CORN MEAL FROM A WHOLE STILLAGE BYPRODUCT AND SYSTEM THEREFORE,” which is incorporated by reference in its entirety for all purposes. There are about forty plants that use a process with a front grinding system (FIG. 2), which are disclosed in the U.S. Pat. Nos. 9,012,191 and 9,689,003, titled “DRY GRIND ETHANOL PRODUCTION PROCESS AND SYSTEM WITH FRONT END MILLING METHOD,” which are incorporated by reference in their entirety for all purposes, which are designed to increase an alcohol yield of the plant as well as to recover valuable oil from the front-end of the process.

The process with a front-end grinding as shown in the FIG. 2 improves the alcohol yield up to 2% and increases oil yield up to 0.9 lb./BU. Nonetheless, such yield of the typical process is still generally less than 50% of its theoretical yields, because there are generally about 1.9 lb/Bu oil in average inside the corn. The low oil yield rate can result from the germ particles still being hard during the short liquefaction stage, which makes it difficult to effectively leach oil into the aqueous phase in the front-end process.

Additionally, the high sugar concentration present in solution in the front-end also exacerbates the limited oil leaching during the front-end process. The improved backend grinding system is developed to address the issues presented above with the front-end grinding, which results in increased oil yield.

The fuel alcohol production originally started using existing wet mill plants and used purified starch to produce alcohol. Because the capital costs of wet grinding mills can be so prohibitive, alcohol plants prefer to use a simpler dry grinding/dry milling process. Around 200 dry milling plants are currently producing fuel alcohol in the U.S. FIG. 1 is a flow diagram of a typical dry grinding ethanol production process 10. As a general reference point, the dry grinding ethanol process 10 can be divided into a front-end and a backend. The part of the process 10 that occurs prior to distillation 14 is referred to as the front-end or front-end process, and the part of the process 10 that occurs after distillation 14 is referred to as the backend or backend process. The definition of front-end and backend processes described above can be applied to the embodiments throughout the Present Specification.

The front-end of the process 10 begins with a grinding step 11, in which dried whole corn kernels are passed through hammer mills to be ground into corn meal or a fine powder. The screen openings in the hammer mills are typically of a size 7 or about 2.78 mm. The resulting particle distribution from the hammer mills generates a very wide, bell type curve particle size distribution, which includes particle sizes as small as 45 microns and as large as 2 to 3 mm. The ground meal is mixed with water to create slurry, and a commercial enzyme with at least alpha-amylase character is added (not shown). This slurry is then heated to approximately 120° C. for about 0.5 to 3 minutes in a pressurized jet cooking process in order to gelatinize (solubilize) the starch in the ground meal. It is noted that in some processes a jet cooker is not used and a longer hold time is used instead.

The grinding step 11 is followed by a liquefaction step 12, wherein ground meal is mixed with cook water to create slurry and a commercial enzyme (e.g., alpha-amylase) is added (not shown). The pH is adjusted to about 4.5 to 6 and the temperature is maintained between 50° C. to 105° C. to convert the insoluble starch in the slurry to soluble starch.

The stream after the liquefaction step 12 has about 30% dry solids (DS) content with all the components contained in the corn kernels, including sugars, protein, fiber, starch, germ, grit, oil and salt. There are generally three types of suspended solids in the liquefaction stream: fiber, germ, and grit. All three of these solids have about the same particle size distribution. The liquefaction step 12 is followed by a fermentation step 13. This simultaneous step is referred to in the industry as “Simultaneous Saccharification and Fermentation” (SSF). In some commercial dry grinding ethanol processes, saccharification and fermentation occur separately (not shown). Both individual saccharification and fermentation and SSF can take as long as about 50 to 60 hours. Fermentation converts the sugar to alcohol using a fermenter. Subsequent to the saccharification and fermentation step 13 is the distillation (and dehydration) step 14, which utilizes a still to recover the alcohol.

In the backend of the process 10 (after a distillation step 14), a fiber separation step 15 (involving centrifuging the “whole stillage” produced at the distillation step 14) to separate the insoluble solids (“wet cake”) from the liquid (“thin stillage”). The “wet cake” includes fiber (per cap, tip cap, and fine fiber), grit, germ particle, and some proteins. The liquid from the centrifuge contains about 6% to 8% DS, which contains mainly oil, germ, fine fiber, fine grit, protein, soluble solid from the fermenter and ash. In some plants, the whole stillage has about 12 to 14% DS that is fed to a first stage evaporator. The whole stillage is concentrated to 15 to 25% DS before feeding to the fiber separation step 15.

At the separator of the fiber separation step 15, the thin stillage flow/portion is split into two streams. A first portion of about 30 to 50% of the flow recycles back (“back-set”) to be mixed with a corn flour in a slurry tank at the beginning of the liquefaction step 12. A second portion of the flow (e.g., the rest of the flow; about 50 to 70% of total flow) then enters the evaporators in an evaporation step 16 to boil away moisture, leaving a thick syrup that contains the mainly soluble (dissolved) solids from fermentation and a dry matter content of between 25% to 45%. The back-set water is used as part of cooking water in the liquefaction step 12 to reduce the amount of fresh water consumption as well as save evaporating energy and equipment costs.

The slurry is able to be subjected to an optional oil recovery step 17, wherein the slurry can be centrifuged to separate oil from the syrup. The oil can be sold as a separate high value product. The oil yield in a traditional, unmodified plant is normally about 0.4 lbs./Bu of corn and that oil contains high free fatty acid. This oil yield recovers only about ¼ of the oil in the corn. About one-half of the oil inside the corn kernel remains inside the germ after the distillation step 14, which cannot be separated in the typical dry grind process using centrifuges. The free fatty acid content, which is partly created when the oil is held in the fermenter for approximately 50 hours, reduces the value of the oil. The common oil recovery centrifuge only removes less than 50% of the oil in syrup, because the protein and oil make an emulsion, which cannot be satisfactorily separated. The adding of chemicals, such as emulsion breaker, can improve the separation efficient in some degrees, but the chemicals are costly and the DDGS product can be contaminated by the added chemicals. Providing heat or raising the feed temperature at the centrifuge to break the emulsion is another way, but it affects the color and quality of DDGS. Adding an alcohol to break the emulsion (U.S. Pat. No. 8,192,627, which is incorporated by reference in its entirety for all purposes) also improves the separation and increases the oil yield, but it needs explosion proof equipment and costly operations. All those improvements only increase the oil yield from an average of 0.4 lbs./Bu to about average 0.6 lbs./Bu even though the “free” oil in the whole stillage is about 1 lbs./Bu. The oil/protein emulsions formed during the whole dry mill process is the main reason for having a low oil yield in the backend oil system.

A backend oil and protein recovery process as shown in FIG. 3 in the patent application (U.S. Patent Application Publication No. 2014/0317945, titled “METHODS FOR PRODUCING A HIGH PROTEIN CORN MEAL FROM A WHOLE STILLAGE BYPRODUCT AND SYSTEM THEREFORE,”) is developed by using an oil/protein separation step 31 that is added to break this oil/protein emulsion on a whole stillage. As shown in the process 30 of FIG. 3, the front-end process is as simple as the existing dry mill process. The whole stillage goes through the fiber separation step 33 to remove the fiber. The dewater fiber is sent to fiber washing step 34 to remove protein using cook water as washing liquid. The combined filtrate (from fiber separation step 33, washing liquid from fiber washing step 34, and liquid from fiber dewater step 32) goes to the oil/protein separation step 31 to break the bonds between oil and protein. The nozzle centrifuges, other types of disc centrifuges, or decanters are normally used for this type of application. The oil/protein emulsion is broken up in a higher G force inside the centrifuge. The oil goes to the light phase (overflow) discharge and protein goes to a heavy phase discharge (underflow), because of the density difference between oil (density 0.9 gram/mL) and protein (1.2 gram/mL). The light phase (overflow) then is fed to an evaporator step 16 to be concentrated to contain 25 to 40% of DS (forming a semi-concentrated syrup). Next, the semi-concentrated syrup is sent to the backend oil recovery system step 17 to recover oil using the backend recovery system. The light phase stream contains less protein, so it has less chance to form oil/protein emulsion. The oil yield with this system can be as high as 0.8 lb./Bu. The de-oil syrup from the backend oil recovery step 17 can further be concentrated in an evaporator to a much higher syrup concentration such as high as 80%+of DS. The de-oil syrup with low protein has substantially lower fouling characteristics in the evaporator, because of the removal of a large portion of the suspended solids normally found in thin stillage. The underflow from oil/protein separation step 31 goes to a protein dewatering step 35 (e.g., using a protein decanter) for protein recovery. The separated protein cake from protein dewatering step 35 with a content of less than 3% oil is sent to a protein dryer (not shown) to produce high value protein meal, which has a protein content of approximately 50%. The protein yield is about 3 lb./Bu. The liquid from the protein dewatering step 35 is sent back to the front-end as a back-set liquid that is used as part of cooking water in the liquefaction step 12.

A process with a front grinding system as shown in FIG. 2 is provided (disclosed in the U.S. Pat. No. 9,012,191 and U.S. Pat. No. 9,689,003, titled “DRY GRIND ETHANOL PRODUCTION PROCESS AND SYSTEM WITH FRONT END MILLING METHOD,” which are incorporated by reference in their entirety for all purposes) to increase an alcohol, oil and protein yield of the plant. In process 20 as shown in FIG. 2, a front grinding system is added during the liquefication step 12. The front grinding system contains a dewatering step 21 and a grinding step 22. The slurry from the liquefication step 12 goes to the dewater device, such as a paddle screen to separate the liquids from solids. The solids contain fiber, grit, and germ particle and are fed to a grinding device, such as a grinding mill or roller mill to break up large particles to release starch and oil from those solid particles. The release of starch can further produce more alcohol in downstream processes and the alcohol yield increases by about 2%. The release of oil will increase the oil yield by around 20% in yield or an increase of about 0.2 to 0.3 lb./Bu. However, about half of germs inside the corn are still not broken apart sufficiently and do not release the oil contained inside to the aqueous environment by this front grinding system.

This lack of release is primarily because the germ particle is still hard and not easy to be broken down during the short contact time with water in the liquefication step 12. These hard germ particles become much softer and much easy to break after the extended soaking time (approximately 50 hours) of the fermentation step 13 as well as the higher temperature experienced during the distillation shown in step 14.

### SUMMARY OF THE INVENTION

In an aspect, the method produces a syrup having a concentration of 80% DS (e.g., dry solid) or higher (e.g., achieved through evaporative concentration) while maintaining a pumpable fluid characteristic. In some embodiments, this substantially higher concentrated syrup can bypass the dryer (e.g., designed not going through a dryer or a high heat environment) and is mixed directly with any dry feed materials, such as materials processed in a dryer in the ethanol plants. The process described herein allows the preservation or preventing the destruction of all heat sensitive water-soluble nutrients, such as vitamin produced from yeasts, in the syrup, such that a more nutritious animal feed ingredient is produced.

FIG. 4 shows a dry milling plant in accordance with embodiments. The dry milling plant uses improved separating and grinding processes in the backend of the plant, instead of at the front-end. In some embodiments, the backend process is referred to the processes and/or steps that are performed after fermentation.

FIG. 4 illustrates a dry milling plant with a backend grinding system, which has higher yields of alcohol, oil and protein than the typical alcohol producing plants. Related U.S. Pat. No. 9,388,475, having a title of “Method of and system for producing oil and valuable byproducts from grains in dry milling systems with a back-end dewater milling unit” is incorporated by reference in its entirety for all purposes.

In the process 400 of FIG. 4, the whole stillage (after distillation step 14, e.g., the distillation step 14 at a distiller of FIG. 1) is fed to the suspended particle separation device/process (step 441) to separate coarse solids 4411 (fiber, germ and grit) from liquids (oil and water and soluble materials) and fine solid particles (gluten and yeast) 4412. The coarse solids 4411 (fiber, germ, and grit) are fed to a particle size reduction device (step 442), such as a grinding mill or a roller mill to break the particles into smaller particles, so that substances (e.g., starch, oil and protein) are released from germ and grit particles. The small germ and grit particles, which have been released from the larger fiber particles are sent to the fiber washing device/process (step 443), so that smaller germ and grit particles are recovered from the larger fibers. To make the water in the facility more efficient, there is an option to use cook water 4431 as the washing liquid. After the step of germ and grit recovery, this washing liquid is sent to the front-end and used as cooking water 4431 for liquefication (not shown, which can be the liquefication step 12 of FIG. 1). The use of the cook water 4431 into the liquefaction step makes the water usage more efficient, which also provides a convenient method to recover the valuable germ and grit particles. The washed fiber from the fiber washing device 443 is sent to the fiber dewatering device/process (step 444) to produce a dewatered fiber fraction, which is low in protein (as low, or lower than 10% protein on a dry matter basis) and low in oil (as low as, or lower than 3% oil on a dry matter basis). This washed and dewatered fiber can be included in cattle feeding rations 4441. Alternately, the washed and dewatered fiber, which contains low protein and low oil, makes it an ideal feedstock for cellulose-to-ethanol processes, often called cellulosic ethanol.

Referring back to the step 441, the liquids and fine suspended solids from the particle size separation are processed in a protein decanter/protein decanting process (step 435) to recover gluten and at least some of the spent yeast bodies (yeast proteins). Cake solids that are recovered by the protein decanter at the step 435 have around 50% protein and around 3% oil in a dry matter basis. This high protein feed 4351 is ideal for monogastric animals including chicken, pork and aquaculture.

The backend grinding system disclosed herein produces higher alcohol yields (up to 2.9 gallons per bushel corn), higher oil yields (up to 1.4 lb. per bushel corn), and approximately 6 pounds per bushel of high protein feed with a concentration of approximately 50% of a dry matter basis. The process also produces high purity fiber for animal feed rations or secondary alcohol production.

In typical ethanol plants, the thin stillage has about 2 to 4% insoluble solid by volume. The high concentration of the suspended solid results from using a low G force fiber decanter centrifuge, which cannot recover fine suspended solids very efficiently. The high suspended solids limit the dry solids content that can be obtained by evaporation to about 40 to 50% on a dry matter basis. The reason that 40 to 50% of solids is the maximum dry matter concentration at the typical ethanol plant is that the insoluble solids in the thin stillage produce a high viscosity inside the evaporator, which causes a low flow and poor wetting of the evaporator tubes, which results in the formation of scale and substantially decreased heat transfer efficiency.

In comparison, the percentage of the insoluble solids by volume in thin stillage from a MSC system (e.g., multi-stage centrifuges protein recovery system) is between 1 to 2%. This thin stillage can be effectively concentrated to around 50 to 60% of dry matter basis. The use of the high G force nozzle centrifuge as applied in the MSC system produces low suspended solids thin stillage, which allows a higher dry solids concentration before reaching the high viscosity limitations in the evaporator.

In accordance with some embodiments, the backend grinding system described herein allows the production of unusually low suspended solids in the thin stillage. This low suspended solid allows substantial reduction in viscosity in the evaporator, which allows for a substantially higher dry matter content in the syrup that is produced in the evaporator. This very high solids syrup can bypass, skip, or avoid the dryer system or a heating process, wherein the high solids syrup can be directly sprayed onto the product as an animal feed or nutrient additive. This substantially reduces dryer operational cost for the animal feed production process.

In some typical cases, the thin stillage from a typical “standard” dry mill ethanol process (FIG. 1) contains 6 to 9% insoluble solid by volume (as measured at 3000G second bucket centrifugation test) and generally is concentrated to 30 to 40% Dry Solids (DS) after the evaporation process. This concentrated material is generally called syrup.

In contrast, in accordance with some embodiments, the thin stillage from a dry mill with the backend grinding process (FIG. 3) contains 2 to 4% insoluble solid by volume and evaporative concentration generally produces 40 to 50% DS (syrup) after the evaporation process. In some embodiments, the thin stillage from a dry mill that has been equipped with the protein recovery process (e.g., the process similar to the one shown in the FIG. 2) containing 1 to 2% insoluble solid by volume and evaporative concentrator will generally produce 50 to 60% DS syrup after the evaporation.

FIG. 4A illustrates a backend high-speed centrifuge incorporated system 400A in accordance with some embodiments. The use of a high-speed centrifuge, such as a nozzle disc stack centrifuge to a backend grinding system, allows the process to have cleaner (lower suspended solids concentration) in the thin stillage. In the process 400A of the FIG. 4A, the whole stillage tank at the step 445A is used as a pre-settlement tank.

The underflow from this tank of the step 445A contains a higher density and larger particle diameter suspended solids. These particles are primarily composed of larger fiber, germ, and grit. These large particles (coarse solids) settle, due to gravity, to the bottom of the whole stillage tank where they can be discharged and transferred to particle separation device/step 441A. The step 441A dewaters the coarse solids. The recovered aqueous phase 4411A from the dewatered coarse solids contains soluble materials along with small suspended particles (fine solids), such as gluten and yeast. The dewatered coarse solids 4412A are sent from the step 441A (particle size separation device) to a particle size reduction device/process at a step 442A, such as using a grinding mill to break down (make smaller) germ and grit particles.

Still at the step 442A, the breaking of the germ and grit releases starch, oil and protein into the aqueous stream. At a step 443A, the ground coarse solids are then sent to a fiber washing device using a washing step to remove fine germ particle, fine grit, oil, starch, and protein from the coarse fiber. The fiber produced using this process is a purer fiber, which contains much lower protein (down to 10 to 15% of protein on a dry matter basis) and much lower oil (down to 3 to 4% oil on a dry matter basis). The aqueous washing liquid phase from the fiber washing (step 443A) containing the separated fine germ, fine grit, oil, starch, and protein are recovered from the coarse solids. This aqueous phase 4431A from the fiber washing (the step 443A) is sent back to the frontend of the ethanol process and used as part of cook water in the mash bill to make the liquefaction recipe. This process allows the recovery of the valuable oil, protein and starch into the next round of fermentation.

Referring back to the step 445A, an advanced aspect of the process 400A includes the use of a high speed disc centrifuge at a step 446A to polish the overflow from the protein decanter at a step 435A. The overflow from the step 446A, called polished thin stillage, has low suspended solids and can be processed through the evaporator/evaporating process 416A to produce a syrup 4161A having 60 to 70% DS (e.g., dry solid or dry matter). The underflow from the disc centrifuge (at the step 446A) is directed to another protein decanter (at a step 447A) to recover fine protein (mainly yeast and germ protein).

Referring to a step 447A, the overflow from a protein decanter is added to the original protein decanter overflow (at the step 435A) and sent back to the high speed disc centrifuge (at the step 446A). This high-speed disc stack centrifuge can remove 80 to 90% insoluble solids per single pass. In contrast, the overflow from a typical protein decanter contains 3 to 6% insoluble suspended solids by volume. With 80 to 90% suspended solid recovered, the disc stack/centrifuge (at the step 446A) overflow (polish thin stillage) normally produces 0.6 to 1.2% insoluble suspended solids by volume allowing the production of 60 to 70% DS (e.g., dry solid or dry solid matter) syrup from the evaporator.

FIG. 4B illustrates a backend double high-speed centrifuges incorporated system 400B in accordance with some embodiments. When a higher than 70% DS syrup concentration is produced, an additional high-speed disc stack centrifuge (step 448B) is added to the process system of FIG. 4A, which is shown in the process 400B of FIG. 4B. As shown in the FIG. 4B, the overflow from the first high-speed disc centrifuge (at a centrifuging step 446B) is sent to a second-high speed centrifuge (at the step 448B) to further polish the thin stillage to produce cleaner thin stillage before being processed in the evaporator (at a step 416B). The underflow from second disc centrifuge (at the step 448B) is combined with overflow from protein decanters (at the step 435B) to be fed to the first high speed disc centrifuge (at the step 446B).

This second disc stack centrifuge (at the step 448B) normally can remove 70 to 80% of the insoluble solid in feed. With 0.6 to 1.2% insoluble solid by volume in feed from the first disc centrifuge (step 446B), the overflow from the second disc stack centrifuge (step 448B) will be approximately to 0.25 to 0.4% insoluble solid by volume. This cleanest thin stillage can be sent to the evaporator (at the step 416B) to produce syrup with 70 to 80% DS.

FIG. 4C illustrates a backend disc decanter centrifuge incorporated system 400C in accordance with some embodiments. In some embodiments, the first high speed disc centrifuge (at the step 446B of FIG. 4B) and the protein decanter (at the step 447B of FIG. 4B) are replaced with a disc decanter centrifuge at a step 449C of the process 400C of FIG. 4C. This disc decanter has higher G force than either a nozzle disc centrifuge or a solid bowl decanter. The overflow has very low suspended solids concentration and the underflow has unusually dry cake.

Referring to a step 416C, the syrup produced at the evaporator has glycerol and residual sugars from the fermentation in the process. These compounds have significantly elevated viscosity when concentrated during syrup evaporation. These materials increase the syrup viscosity significantly and sharply when the syrup concentration is over 50% of DS. A similar syrup enriching process, disclosed in the PCT/US2016/038436, titled “A METHOD OF AND SYSTEM FOR PRODUCING A HIGH VALUE ANIMAL FEED ADDITIVE FROM A STILLAGE IN AN ALCOHOL PRODUCTION PROCESS,” is incorporated by reference in its entirety for all purposes, which can convert those materials to organic acids, such as lactic acid. These organic acids have lower viscosity than glycerol or sugars and such conversion decreases the final syrup viscosity allowing to produce a much higher syrup concentration.

Accordingly in some embodiments, the process 400C uses the methods and system disclosed above to convert glycerol or sugars to organic acids at the evaporator 416C.

FIG. 5 illustrates an enriched syrup enhancing process 500 in accordance with some embodiments. As shown in a process 500 in (FIG. 5), the enriched syrup (at a step 551) is added to the evaporator during the evaporating (at a step 516). Because of the lower viscosity of the organic acids, the final syrup concentration can reach 80 to 90% DS.

The process 500 of FIG. 5 illustrates a dry milling ethanol plant using high speed centrifuges in series for performing a series of centrifugation in accordance with some embodiments. The series of centrifugation steps 5461 include a step 546 (e.g., using a disc centrifuge), a step of 547 (e.g., using a protein decanter centrifuge), and a step of 548 (e.g., using a disc centrifuge)) to polish a thin stillage. Next, the enriched syrup forming process (at the step 551) is used to produce a concentrated syrup with more than 80% DS. This very high solids contained syrup can be added directly to DDG and protein meal after the coarse solids that are dried by a dryer (not show), which forms a value added material, such as an animal feed.

This advantageous feature allows the plant/factory to avoid subjecting the syrup to high heat in the dryer. High heat can destroy heat sensitive nutrients inside the syrup. In some embodiments, the process disclosed herein also substantially decreases the dryer temperature to almost half of what it is in the traditional process. This advantageous feature allows the manufacturing plant to divide a series of dryers into two parallel dryers lines allowing the production of a high and low protein feed. In some embodiments, the dryers are performed concurrently, so that one dryer is drying a first portion of the input and another dryer is drying a second portion of the input. For plants that have multiple independent dryer systems, the plant can now devote those different dryers making a low protein product from one set of dryer(s) and making a high protein product using another set of dryer(s) in the system(s). This is a significant efficiency improvement and capital savings technology for the plants.

FIGS. 6, 6A, and 6B illustrate a different arrangement of the centrifuges 600, 600A, and 600B (e.g., high-speed disc stack centrifuges and decanters) in accordance with some embodiments. The two high-speed disc stack centrifuges (such as step 646 and 648) and two protein decanters (step 635 and 647) are arranged in various ways, sequences, and are able to be used directly next to each other or independently anywhere in the process disclosed herein. In some embodiments, the disc centrifuges and decanters are arranged in any orders and in any locations in the system/process.

FIG. 7 illustrates a combined feature process 700 in accordance with some embodiments. The process 700 of FIG. 7 is the combination of a) backend grinding steps 741, 742, 743, and 744 (Group I Process), b) using a clean thin stillage system with two-disc centrifuges (step 746 and 748) and two protein decanters (step 735 and 747), which is referred to as Group II Process, c) adding an enriched syrup at the step 751, which is referred to as Group III Process, and d) splitting two dryers that are currently operated in series into two independent parallel dryers allowing, one dryer for protein dryer (step 771) and one DDG dryer for DDGS (step 772), which is referred to as Group IV Process. Any of the groups and processes described in the FIG. 7 are able to be selected to be used or omitted in some embodiments. This process 700 forms one of the optimized dry milling process, which increases the ethanol yield up to 3%, produces up to 1.4 lb./Bu oil yield, and produces up a protein meal yield to 6 lb./Bu of corn with up to 50% protein content.

### BRIEF DESCRIPTION OF THE DRAWINGS

FIG. 1 is a flow diagram of a typical dry milling process and system for producing ethanol and recovering oil in a backend process.

FIG. 2 is a flow diagram of a typical dry milling process and system with a front-end grinding to increase the alcohol and oil yield.

FIG. 3 is a flow diagram of a typical dry milling process and system for producing ethanol and recovering oil and protein at the backend.

FIG. 4 is a flow diagram of a system for and method of a dry milling process with a backend grinding process and system for increasing the alcohol and oil yields in accordance with some embodiments.

FIG. 4A is a flow diagram of a system for and method of a dry milling process with a backend grinding and one-disc centrifuge to polish the thin stillage in accordance with some embodiments.

FIG. 4B is a flow diagram of a system for and method of a dry milling process with a backend grinding and two-disc centrifuges in series to polish thin stillage in accordance with some embodiments.

FIG. 4C is a flow diagram of a system for and method of a dry milling process having a backend grinding with a disc decanter in accordance with some embodiments.

FIG. 5 is a flow diagram of a system for and method of a dry milling process with a backend grinding, two-disc centrifuges in series, and a secondary fermentation process and device to produce a high % DS syrup in accordance with some embodiments.

FIG. 6 is a flow diagram of a variation of the system and method described in FIG. 5 in accordance with some embodiments.

FIG. 6A is a flow diagram of a variation of the system and method described in FIG. 5 in accordance with some embodiments.

FIG. 6B is a flow diagram of a variation of the system and method described in FIG. 5 in accordance with some embodiments.

FIG. 7 is a flow diagram of a system for and method of a dry milling process with a) a backend grinding to increase alcohol, oil, and protein yield, b) two-disc centrifuges in series to produce cleanest thin stillage, c) a secondary fermentation to produce enriched syrup with the highest % DS syrup, d) splitting two dryers in series to two dryers in parallel to produce high protein meal and enriching low protein feed in accordance with some embodiments.

FIG. 7A is a flow diagram of a variation of the system and method described in FIG. 7 in accordance with some embodiments.

FIGS. 8A-8E illustrate experimental results in accordance with some embodiments.

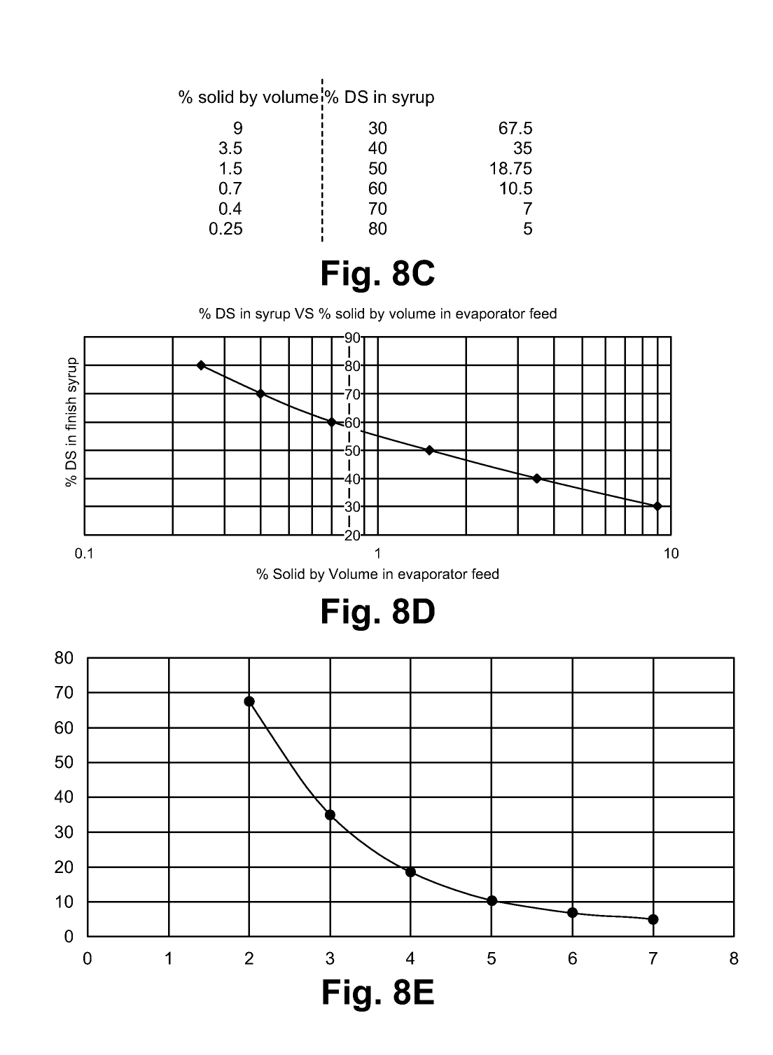

### DETAILED DESCRIPTION OF PREFERRED EMBODIMENTS

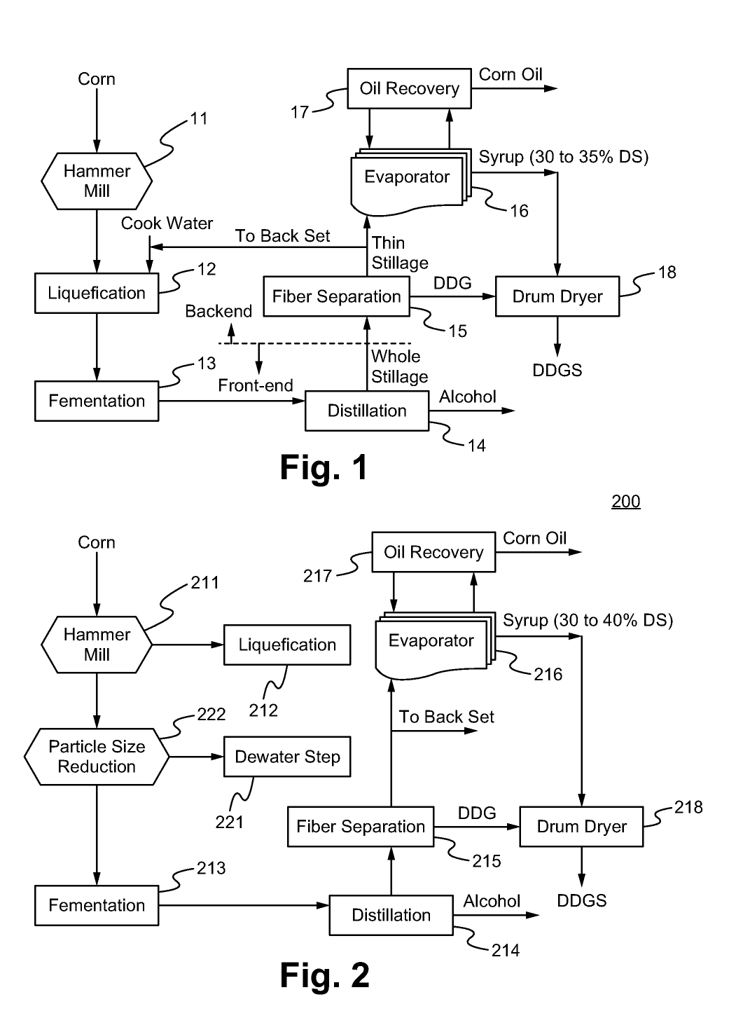

FIG. 1 illustrates a typical dry milling process, where corn is processed through a hammer mill (step 11) to break the whole kernels into smaller particles (less than 2.78 mm). Cook water and enzyme is added to the ground corn to liquefy (step 12) the starch. Fermentation is conducted with the addition of yeast to the liquefied corn slurry to convert starch to alcohol in fermentation (step 13). After approximately 30-70 hours fermentation time, the finished beer (generally containing 12% or higher alcohol) is sent to the distillation tower (step 14) to recovery the alcohol produced. The whole stillage from the bottom of the distillation tower (step 14) is send to the decanter (step 15) to remove coarse particles including fiber and large particles of protein, germ and grit for the recovery as a DDG cake. The overflow (thin stillage) from fiber separation (step 15) contains mainly fine particles of protein, germ, oil, oil emulsion and water soluble materials and is sent to the evaporator (step 16) to be concentrated to become syrup containing 25%-40% of DS.

An oil recovery (step 17) process has been added to the majority of dry milling plant to recover valuable oil during the evaporation process. The syrup can be concentrated to have 25%-40% of DS depending on the capacity and capability of the decanter and evaporator and the % insoluble solid present (measured by volume in a lab centrifuge at 3000G seconds spin) in the thin stillage. The majority of dry mill ethanol plants has 6 to 12% insoluble solids by volume in the thin stillage and produces a 30 to 40% DS syrup.

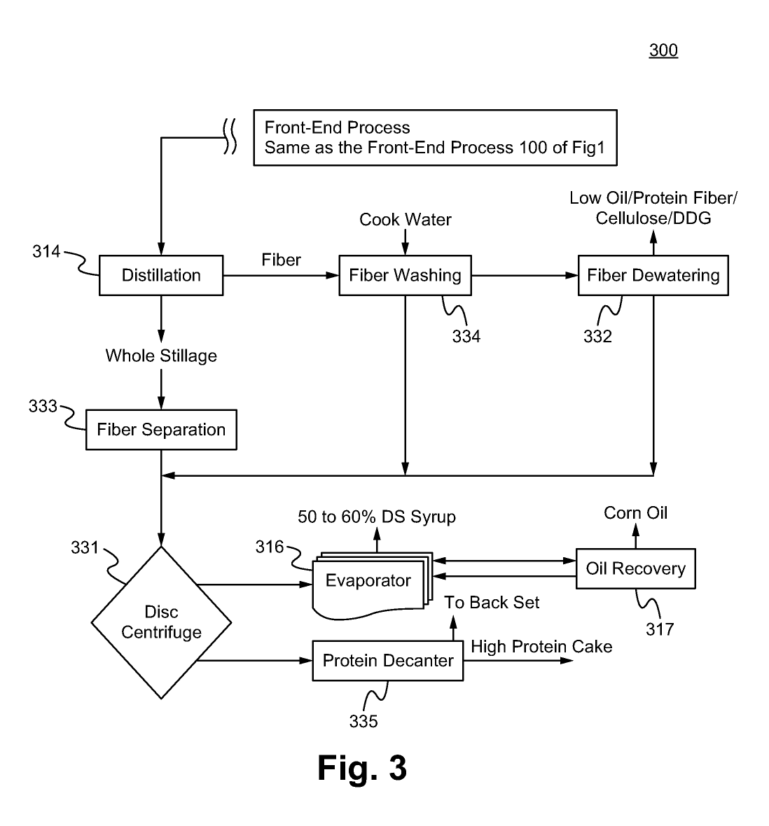

FIG. 2 shows a typical dry milling process and system with front-end grinding to increase the alcohol and oil yield. As shown in the process 200 of FIG. 2, solids dewatering (step 221) and particle reduction device (step 222) is added to the process 100 of FIG. 1 to form the process 200 (FIG. 2).

The whole kernel grains (often maize, corn) go through a hammer mill (step 211) to break up the kernels into smaller particles (less than 2.78 mm). Cook water and enzyme are added to the ground corn in order to liquefy (step 212) the starch. Before sending the liquefaction to the fermenter, the liquefied slurry is sent to the solid dewatering device (step 221) to remove majority of liquid and then send the relatively dry solids to a particle reduce device (step 222) to break up large grit and germ particles to increase the alcohol and oil yield. The combined liquid from solid dewatering process (step 221) and the ground solids from the particle size reduction device (step 222) is then added to the fermenter (step 213) along with yeast to convert starch to alcohol in the fermentor for a fermentation process (step 213). After approximately 30-70 hours of fermentation time, the finished beer (generally containing 12% or higher alcohol) is sent to the distillation tower (step 214) to recover the alcohol.

The whole stillage from the bottom of the distillation tower (step 214) is sent to the decanter (step 215; a fiber separating process) to remove coarse particles including fiber and large particles of protein, germ and grit for the recovery as DDG cake. The size of solid in the whole stillage after the front-end grinding step in the process 200 will decrease, but those reduced particle size solids will still be recovered in its majority in the decanter's recovery range (step 215; generally down to 5 to 10 micron in their diameter). This results in a percentage of the insoluble solid ranging from 6% to 12% in the thin stillage of the process 200, which has an insoluble solid percentage almost identical to the process 100. Thus, the overflow (thin stillage) from the fiber separation (step 215) contains mainly fine particles of protein, germ, oil, oil emulsion and water soluble materials and is sent to the evaporator (step 216) to be concentrated to have a syrup of about 25 to 40% DS. An oil recovery process (step 217) has been added to the majority of the dry mill plant to recover valuable oil during the evaporation process. The syrup can be concentrated to 25 to 40% of DS depending on the capacity and capability of the decanter and evaporator and the percentage of the insoluble solid present (measured by volume in a lab centrifuge at 3000G seconds spin) in the thin stillage. The majority of the dry mill ethanol plants has 6 to 12% insoluble solids by volume in thin stillage and produces a syrup with 30 to 40% DS (dry solid).

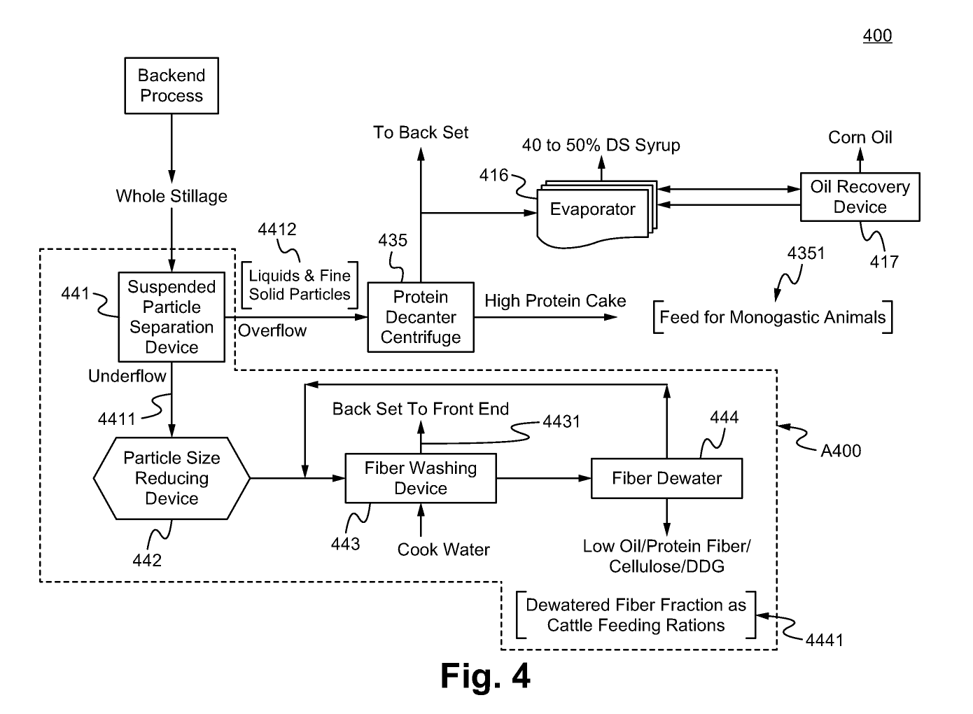

FIG. 3 is a flow diagram of a typical dry milling process and system for producing ethanol and recovering oil and protein at the backend. As shown in process 300 of FIG. 3, the fiber separation (step 333), fiber washing (step 334), fiber dewatering (step 332) and a thin stillage clarification processes (step 331; disc centrifuge) are additional to the typical dry mill process 100 of FIG. 1 to separate/recover protein and produce high protein meal. In the process 300 of FIG. 3, the whole kernel grains (often maize, corn) go through a hammer mill to break up the kernels into smaller particles (less than 2.78 mm). Cook water and enzyme are added to the ground corn to liquefy the starch. The liquefaction is then added to the fermenter along with yeast to convert starch to alcohol in a fermentor (step 13; fermenting). After approximately 30-70 hours of fermentation time, the finished beer (generally containing 12% or higher alcohol) is sent to the distillation tower (step 314; distilling) to recover the alcohol. The whole stillage from bottom of the distillation tower (step 314) is sent to a fiber separation process (step 333) to remove the fiber, the fiber portion goes to fiber washing process (step 334) to remove/recover protein. After washing, the washed fiber goes to the fiber dewatering process (step 332) to produce low protein and low oil DDG/cellulosic fiber. The liquid from fiber separation (step 333), the fiber washing (step 334) and the fiber dewatering (step 332) are combined to be sent to a high speed disc stack centrifuge (step 331) to break the emulsion bond between the oil and protein in the aqueous phase. The overflow (thin stillage) from disc centrifuge contains most of the oil and emulsion along with soluble compounds and is sent to the evaporator (step 316) and oil recovery (step 317) processes to produce high concentration syrup. Because of higher (6000 to 8000 G) G force on the disc stack centrifuge (step 331), the thin stillage only contains about 1 to 2% insoluble solid by volume. This is substantially better as compared with the overflow (thin stillage) from low G force (3000 to 4000G) fiber separation decanter (step 15) in process 100 of FIG. 1 and the step 215 of process 200 of FIG. 2. The percentage of DS produced by evaporation using the process 300 is normally in the range of 50 to 60% as compared to 30 to 40% DS syrup in the process 100 of FIG. 1 and process 200 of FIG. 2.

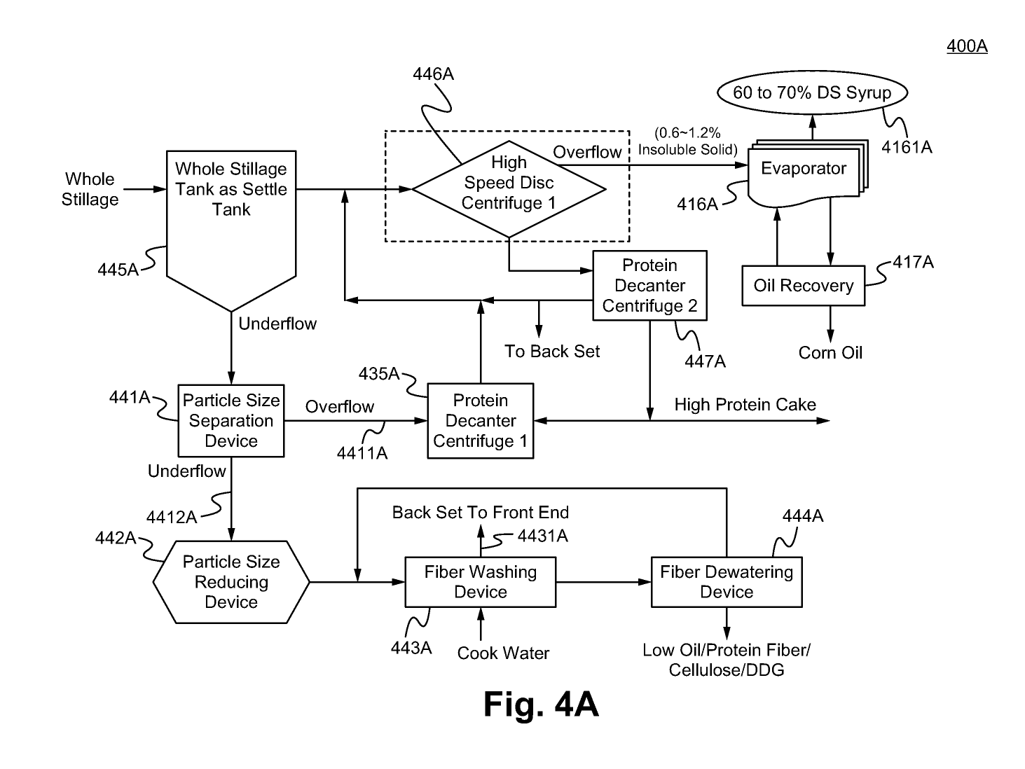

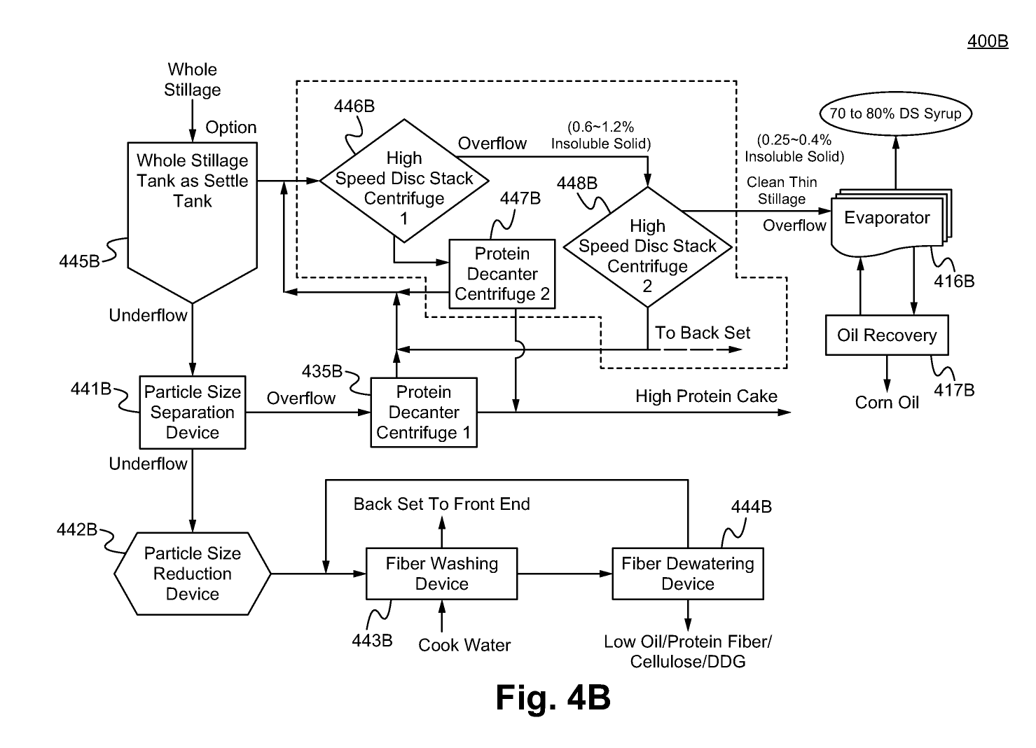

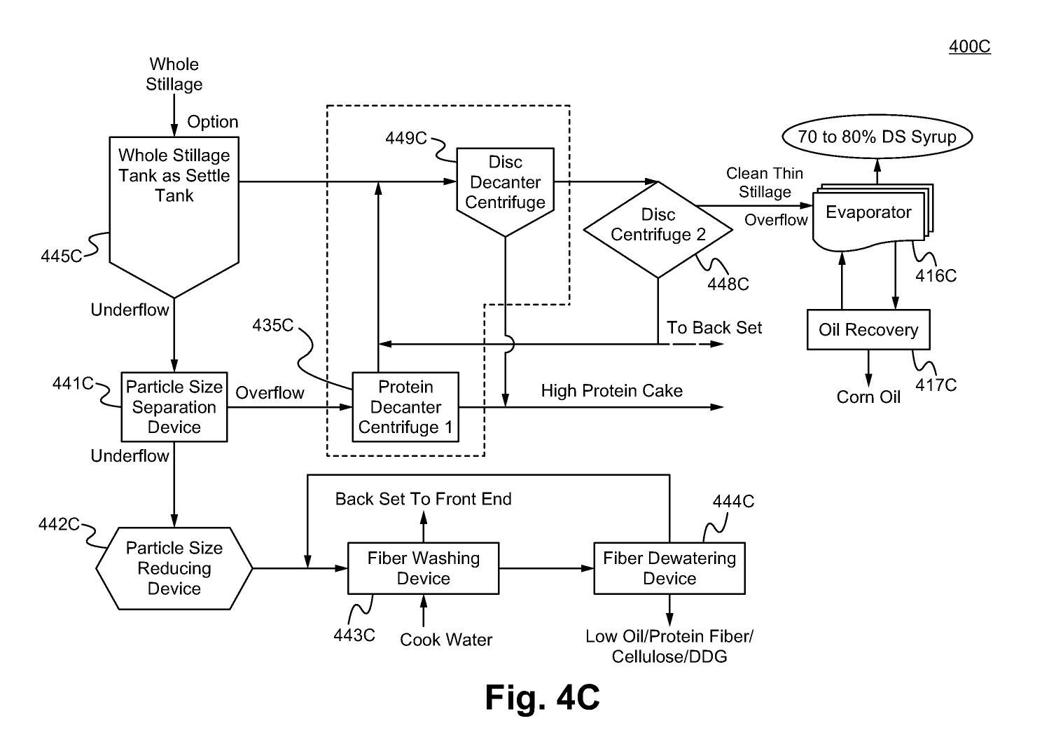

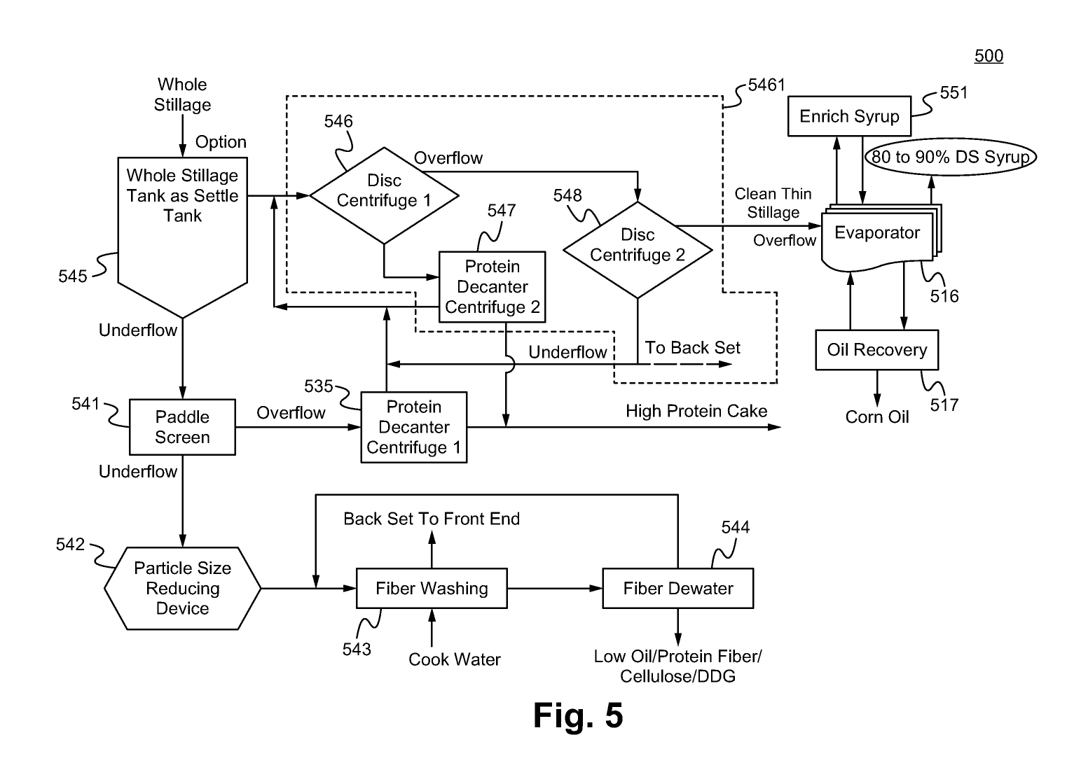

FIG. 4 is a flow diagram of a system for and method of a dry milling process with a backend grinding processes and systems A400 for increasing the alcohol and oil yields in accordance with some embodiments. As shown in the process 400 of FIG. 4, a backend grinding system (step 441, particle size separation) and a particle size reducing device (step 442), a fiber washing process (step 443) and a fiber dewatering process (step 444)) are added to the typical dry mill process 100 of FIG. 1.

In the process 400, the front-end (process before distillation) is the same as a typical dry mill ethanol process 100 of FIG. 1. The whole stillage from distillation bottom is sent to the particle size separation (step 441) to separate the coarse solids (large fiber, germ, and grit) from fine solids (small diameter protein, yeast), oil, and emulation with soluble materials. The coarse solids are sent to a particle reduce device (step 442) to break down the large germ and grit particle and release the oil, protein and starch. The particle size separation process (step 441) and particle size reduction device step (step 442) are more advanced than the dewatering (step 221) and particle size reduction device (step 222) in process 200 of FIG. 2.

The paddle machine or any similarly performing screening device can be used in the process for particle size separation (step 441). The screen size normally has a range of between 50 to 300 microns depending on the type of screen (including slotted or round hole) and the purity of the protein meal desired. The germ particles and grit particles are much softer and easier to break after the extensive soak time during fermentation in combination with the heat and violent agitation in the distillation. A wide range of particle reducing devices from high intense grinding mill and to low HP (horse power) consumption roller mill can be used.

The ground solids from the particle reduce device (step 442) are sent to the fiber washing (step 443) to remove protein, fine germ, and starch using cook water as washing liquid 4431. This washing liquid 4431 picks up the protein, fine germ particle, oil, and starch from the ground fiber and send this valuable material back to the front-end as part of cooking liquid, thus recovering these components during the next round of fermentation batch. The washed fiber is sent to fiber dewatering (step 444) to get dry fiber cake with low protein and low oil, which can be sold as DDG product or further processed to cellulosic ethanol.

The filtrate from particle size separation (the step 441), which contains fine particles including protein and yeast is sent to the existing protein decanter (the step 435) to recover protein and produce high protein cake. Depending on the protein yield that is needed and the capacity of the protein decanter, the overflow (thin stillage) from the protein decanter normally ranges between 3 to 6% of insoluble suspended solid by volume. This material is sent to the evaporator (the step 416) to produce 40 to 50% DS syrup depending on the type of evaporator and capacity of evaporator. Some of overflow from protein decanter used as backset (e.g., water/solution) to save energy.

If a higher percentage of DS syrup is needed, the addition of a high speed disc stack type centrifuge (e.g., nozzle centrifuge, desludger centrifuge, and disc decanter) to polish the thin stillage (remove additional suspended solids) to get cleaner thin stillage can be applied, as shown in the process 400A of FIG. 4A.

FIG. 4A is a flow diagram of a system for and method of a dry milling process with a backend grinding and one-disc centrifuge to polish the thin stillage in accordance with some embodiments.

In the process 4A, the whole stillage tank can be used as gravity, pre-settling tank to avoid making excess emulation in the down-stream processing step while improving the separation efficiency and obtaining higher oil and protein yields. The whole stillage is transferred from the beer column bottom and goes to the whole stillage tank. This specification provides that the whole stillage tank can be used as a gravity pre-settling vessel (step 445A). The overflow from the whole stillage tank (step 445A) contains mainly oil, emulsified oil, and fine particle size protein. This material is sent to a high-speed disc stack style centrifuge to break the bond between the oil and protein to produce a) overflow containing primarily oil and emulsion, and b) underflow containing primarily protein slurry and decreased oil concentration. The overflow from the disc centrifuge (step 446A) is sent to the evaporator (step 416A) and oil recovery (step 417A) and can produce higher concentration syrup because of the removal of suspended solid particles.

Insoluble particles are removed by the high-speed, disc style centrifuge which can recover 70 to 90% of the insoluble solid depend on capacity and style of the disc centrifuge. Normally the percent (%) concentration of insoluble solid in thin stillage from a disc centrifuge has a range of 0.6 to 1.2% by volume. Thin stillage with this concentration of insoluble solid can produce 60 to 70% DS syrup in the typical evaporator systems of an ethanol plant. The underflow from the disc centrifuge (step 446A) is sent to a new protein decanter (step 447A) to produce high concentration protein meal, which is enriched in yeast and germ protein.

The underflow from the whole stillage tank (step 445A) operating in a gravity, pre-settling mode (step 445A) has much higher concentration of coarse solids (large fiber, germ and grit particles) along with some fine protein. This stream can go to particle size separation (step 441A) to preferentially separate fine protein suspended solids and other fine suspended solids from those coarser solids. After separation, the liquid that contains those fine suspended particles (including fine protein solid) is sent to a protein decanter (step 435A) to recover protein and produce one or more high concentration protein meal cakes with between 42 and 55% protein content on a dry matter basis. The overflow from both protein decanters (step 435A and step 447A) is combined with the overflow from whole stillage tank (step 445A) to feed the disc style centrifuge (step 446A). Some overflow from protein decanter (step 447A) is used as backset to save energy. The underflow from coarse particle separation (step 441A) is processed by the particle size reduction device (step 442A).

After particle size reduction, the fiber washing (step 443A) and fiber dewatering (step 444A) processes produce a low protein and low oil DDG that can be used as animal feed or further processed into cellulosic ethanol using the same process 40 shown in FIG. 4.

FIG. 4B is a flow diagram of a system for and method of a dry milling process with a backend grinding and two-disc centrifuges in series to polish thin stillage in accordance with some embodiments.

In order to produce the cleanest thin stillage (lowest suspended solids), which allows one to produce the highest % DS syrup, the use of one additional high speed disc style centrifuge can be used to further polish the thin stillage.

As shown in a process 400B, the second high speed disc style centrifuge (step 448B) is added to the process 400A (FIG. 4A) to further polish the overflow from first disc style centrifuge (step 446B). The overflows from disc style centrifuge (step 448B) is sent to the evaporator allowing the production of the highest concentrated syrup. The percentages of the insoluble solid by volume in the overflow from the second disc centrifuge (step 448B) is in the range of 0.2 to 0.6% allowing the production of 70 to 80% DS syrup. The rest of process steps in process 400B (FIG. 4B) is the same as those previously shown in process 400A (FIG. 4A) in some embodiments.

FIG. 4C is a flow diagram of a system for and method of a dry milling process having a backend grinding with a disc decanter in accordance with some embodiments. In some embodiments, the first disc centrifuge (step 446B of FIG. 4B) and second protein decanter (step 447B of FIG. 4B) in process 400B can be replaced by a disc decanter (step 449C) as shown in process 400C of FIG. 4C. A disc decanter uses less electrical energy while providing higher G force and producing cleaner thin stillage. The rest of process steps in process 400C (FIG. 4C) are the same as those previously shown in process 400B (FIG. 4B) in some embodiments.

The percentage of the insoluble solids in the thin stillage is one of the factors that increases the syrup viscosity. The residual sugars and glycerol present in the syrup increases the syrup viscosity under a high solid concentration. The enriched syrup process (disclosed in the U.S. Patent Application Publication No. 2016/0374364, and titled “A METHOD OF AND SYSTEM FOR PRODUCING A HIGH VALUE ANIMAL FEED ADDITIVE FROM A STILLAGE IN AN ALCOHOL PRODUCTION PROCESS,” which is incorporated by reference in its entirety for all purposes) can be used to decrease the syrup viscosity by using microorganisms to convert sugar and glycerol present in the syrup to organic acids, including lactic acid.

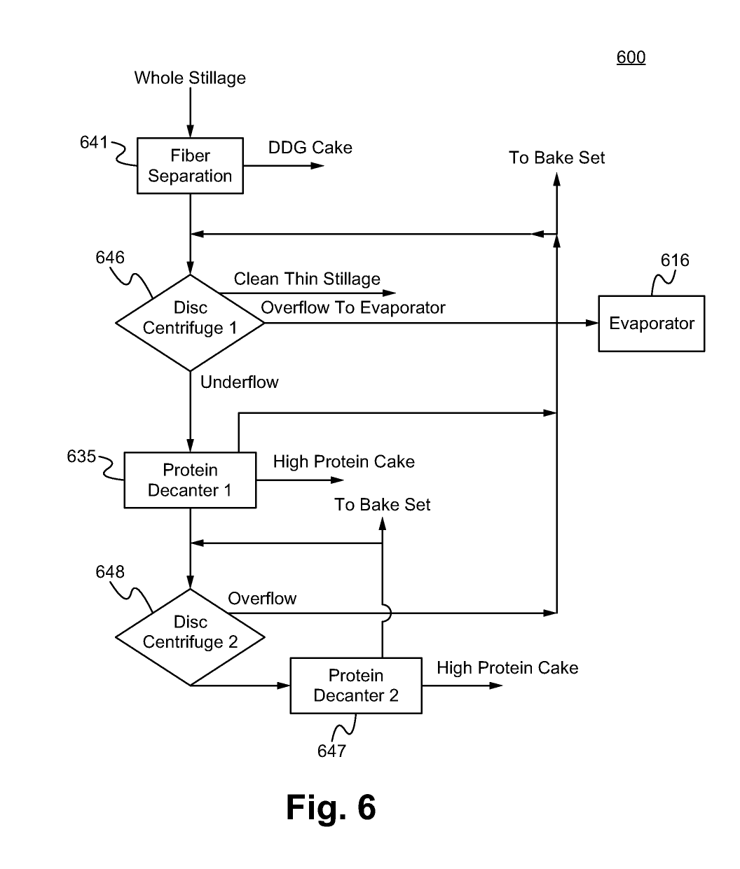

FIG. 5 is a flow diagram of a system for and method of a dry milling process with a backend grinding, two-disc centrifuges in series, and a secondary fermentation process and device to produce a high % DS syrup in accordance with some embodiments. As shown in the process 500 (FIG. 5), the enriched syrup (step 551) can be carried out during the evaporation process (step 516). The enriched syrup (step 551) process not only converts sugars and glycerol to organic acids (including lactic acid), it also serves to break the oil/protein/water emulsion, which improves and enhances protein and oil recovery. The rest of process steps in process 500 (FIG. 5) is the same as those previously shown in process 400B (FIG. 4B) in some embodiments.

Similar to the process 400B (FIG. 4B), the process 500 of FIG. 5 uses a combination of two-disc style centrifuges (step 546B and step 548B) and two protein decanters (steps 535 and step 547) to polish thin stillage before sending to the evaporator (step 516).

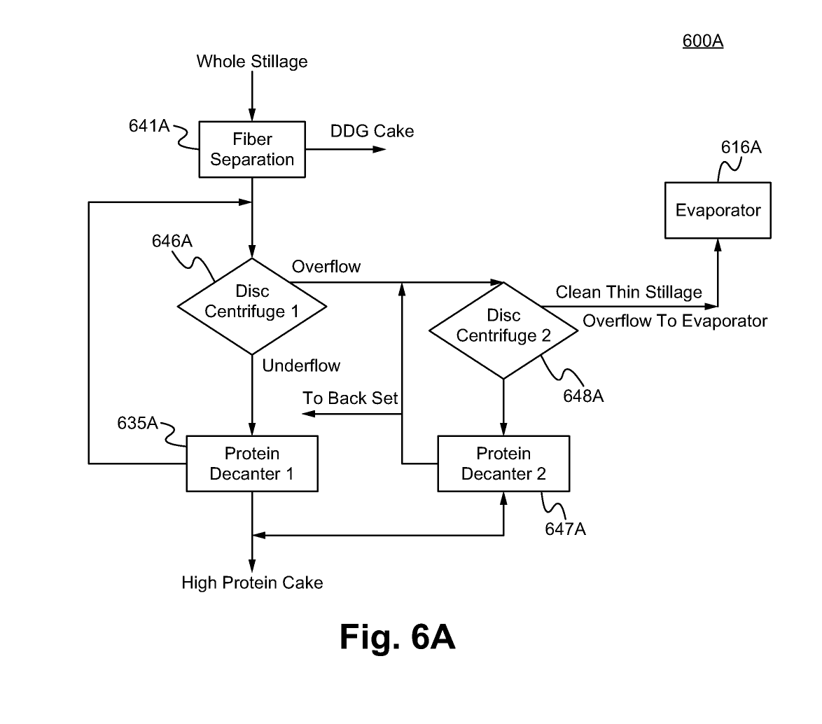

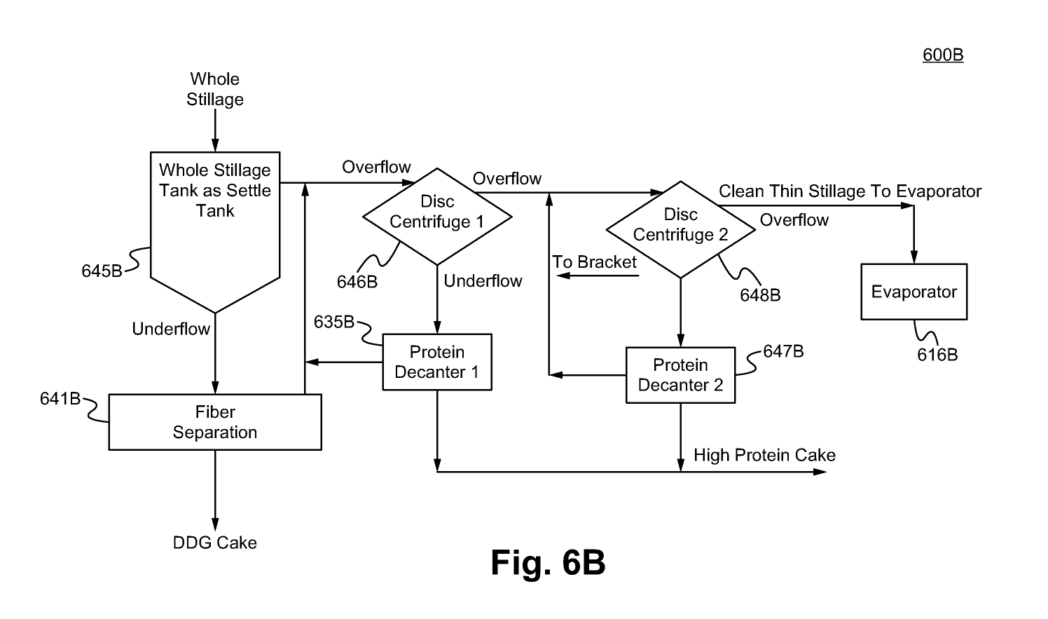

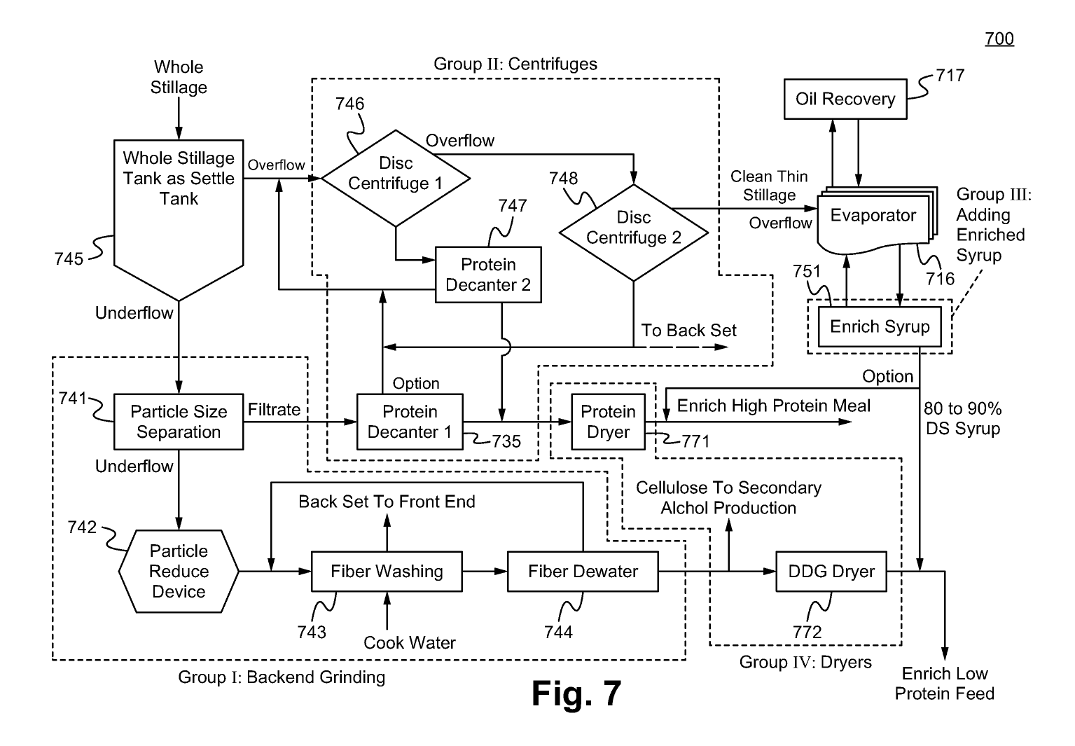

There are several ways to arrange or combine these four steps together. For example:
a) As shown in a process 60 of FIG. 6, the whole stillage is processed through fiber separation (step 641) to remove coarse solids including fiber. The filtrate from the fiber separation (step 641) is processed in the disc style centrifuge (step 646) to produce clean overflow (containing mainly oil and emulsion) for processing in the evaporator (step 616). The underflow from the disc style centrifuge is processed in the protein decanter (step 635) to produce a high protein meal cake. The overflow from the protein decanter 1 (step 635) is processed in disc style centrifuge 2 (step 648) to break up the emulsion bond between oil and protein. The overflow from disc centrifuge 2 (step 648) is primarily oil and some remaining emulsion which is sent back to feed disc style centrifuge 1 (step 646). The underflow from disc centrifuge 2 (step 648) is mainly protein and is sent to protein decanter 2 (step 647) to recover protein. The overflow from protein decanter 2 (step 647) is used as backset to save energy.
b) In the process 60A of FIG. 6A, the whole stillage is processed in the fiber separation process (step 641A). The filtrate from the fiber separation (step 641A) is processed in the disc style centrifuge 1 (step 646A). The overflow, enriched in oil and emulsion, from the disc style centrifuge 1 (step 646A) is processed in the disc style centrifuge 2 (step 648A). The overflow from the disc style centrifuge 2 (step 648A) is processed in the evaporator 616A enabling very high dry solids in the final syrup. The underflow from the disc style centrifuge 1 (step 646A) is sent to the protein decanter 1 (step 635A) to produce a high concentration protein meal cake. The overflow from the protein decanter 1 (step 635A) is recycled back to the disc centrifuge 1 (step 646A). The underflow from disc centrifuge 2 (step 648A) is sent to the protein decanter 2 (step 647A) to recover additional high concentration protein meal cake. The overflow from protein decanter 2 (step 647A) is recycled back to the disc style centrifuge 2 (step 648A) or used as backset to save energy.
c) In the process 60B of FIG. 6B, the beer bottoms are fed to the whole stillage tank (step 645B). In this process the whole stillage tank (step 645B) is used as a gravity pre-settling tank. The overflow from the whole stillage gravity pre-settling tank (step 645B) is enriched in oil and emulsion. This overflow stream is sent to the disc style centrifuge 1 (step 646B). The overflow from disc style centrifuge 1 (step 646B) is sent to disc style centrifuge 2 (step 648B) and the overflow from disc style centrifuge 2 (step 648B) is sent to the evaporator (step 616B). The underflow from disc style centrifuge 1 (step 646B) is sent to protein decanter 1 (step 635B) to produce high concentration protein meal cake. The overflow from protein decanter 1 (step 635B) is recycled back to disc centrifuge 1 (step 646B) as a feed material. The underflow from the disc style centrifuge 2 (step 648B) is sent to a protein decanter 2 (step 647B) to produce high concentration protein meal cake. The overflow from protein decanter 2 (step 647B) is recycled back to disc centrifuge 2 (step 648B) as a feed material. Some of overflow from protein decanter used as backset to save energy.

A person of ordinary skill in the art appreciates that there are many ways to arrange the two-disc style centrifuges (step x46y and step x48y) and the two protein decanters (step x35y and step x37y) to produce very clean thin stillage for feed to the evaporator while also producing dry, high purity protein cake from whole stillage. The x and y of the x46y, x48, x35, x 37 represent the respective machine and device in a figure, such as x=6 and y=B when the centrifuge is in the FIG. 6B as the centrifuge 646B.

In some embodiments, the systems and devices are used to produce the cleanest thin stillage with a readily available and reliable centrifuge technology. The very low suspended solid thin stillage allows the production of a very high concentration of a dry matter basis in the syrup. The advantages of producing this very high solids syrup concentration are numerous, including: a) cutting down the dryer load; b) removing as much water as possible in the evaporator to save energy in the operation of the dryer; c) recovering clean water that can be reused in the ethanol process thereby reducing input water demand; d) producing a very high syrup concentration, (e.g., 80% DS). The syrup can bypass the dryer entirely allowing the maximum syrup concentration to be added after the dryer; e) avoiding sending the syrup to the dryer, which allows the avoidance of heat sensitive nutrient and probiotic in syrup to be damaged by the high temperatures that are often experienced in the distiller's dryers; and (f) when syrup is dry enough to bypass the dryer, the load on an existing dryer system is substantially reduced. This creates the possibility of modifying the typical two drum dryers in series to become two drum dryers working in parallel operation. This allows for one dryer to now produce DDG (low protein feed) and the other dryer to produce a high protein meal. This advantage allows an existing facility to diversify their products without the substantial capital spending for new dryers.

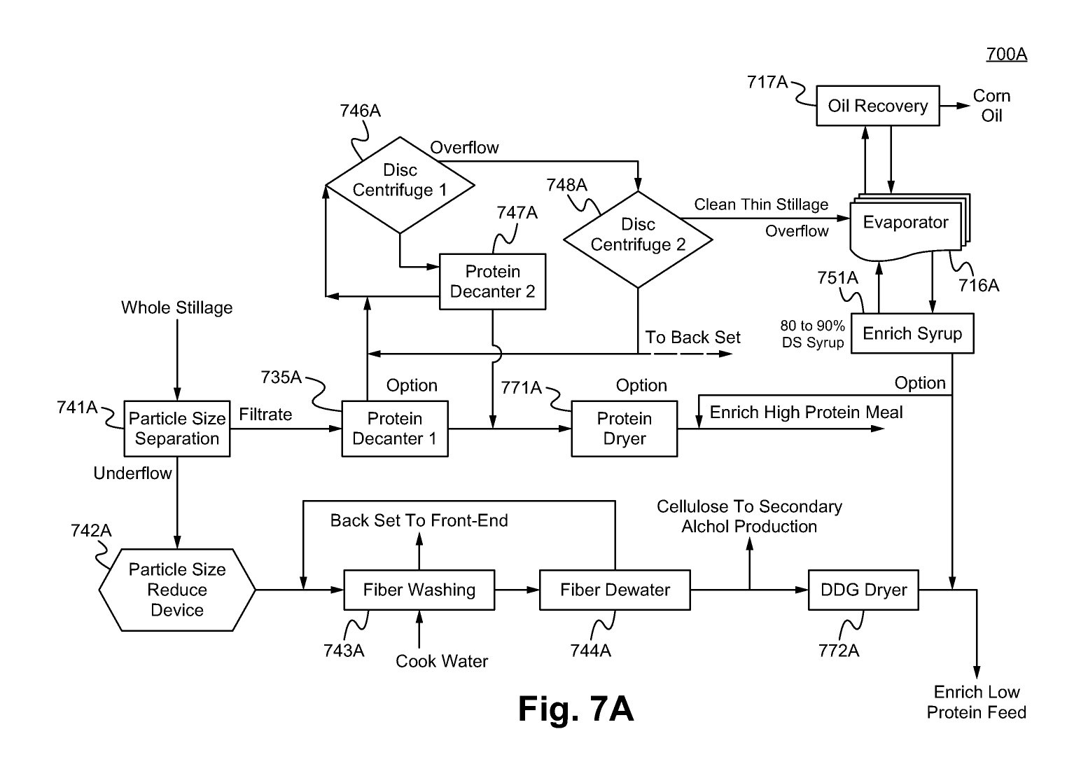

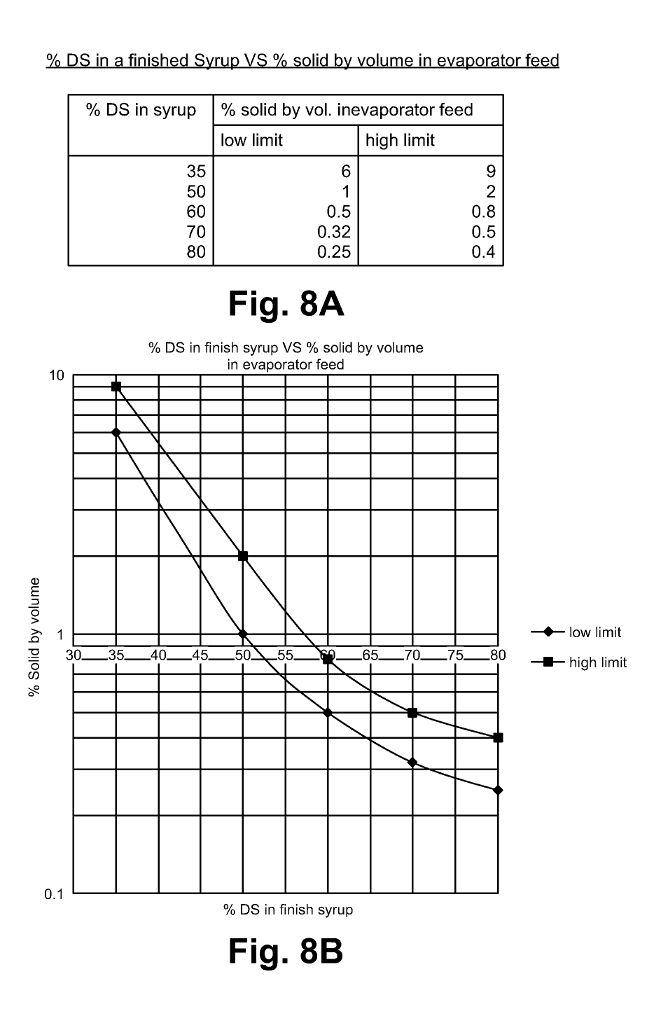

In some embodiments, the process 700 of FIG. 7 is applied to a two-drum dryer that has been modified to operate in parallel into the process 50 (FIG. 5). This process produces a substantially enhanced and more efficient dry milling ethanol process for the typical dry mill industry. In the process 700 of the FIG. 7, the whole stillage is fed to the whole stillage tank (step 745), which is used as gravity, pre-settling tank (step 745A). The overflow from the pre-settling whole stillage tank (step 745A) is fed to the disc style centrifuge 1 (step 746) to break the bond/interactions forming undesired emulsion between oil and protein. The overflow from disc style centrifuge 1 (step 746) is enriched in oil and emulsion with small amounts of fine suspended particles (including protein). This overflow is sent to the disc style centrifuge 2 (step 748) to polish and remove many of the fine suspended particles, including protein. The overflow from disc style centrifuge 2 (step 748) is sent to the evaporator (step 716). A semi-concentrated syrup, around 20 to 40% of DS, is processed through the common oil recovery processes (step 717) to recover and enriched syrup (step 751) to convert organic materials, including sugars and glycerol to organic acids, including lactic acid. This conversion to organic acids decreases the syrup viscosity. The final syrup concentration produced by the evaporator can reach 80% of DS or more with this process. The underflow from the whole stillage tank (step 745) is processed through the particle size separation operation (step 741). The underflow from the particle size separation (step 741) is directed to the particle size reduction device (step 742). The material exiting the particle size reduction (step 742) is processed through the fiber washing (step 743) to wash out fine particles including germ and grit particles.

These recovered fine particles are recovered with cook water which will be returned to the front-end of the ethanol plant. This washing liquid which now contains fine germ and grit particles is sent back to the front of the plant, which results in the increase in alcohol, oil, and protein yield. The washed cake from the fiber washing (step 743) process is sent to a fiber dewatering (step 744) to produce dry DDG cellulose cake. This cellulose cake can be further processed into cellulosic ethanol. The liquid from the fiber dewatering step is recycled to the fiber washing (step 743) to recover more valuable fine particles from the ground fiber. The filtrate from the particle size separation (step 741) process is processed in the protein decanter 1 (step 735) to produce a high purity protein cake. The overflow from the protein decanter 1 (step 735) is combined with the underflow from disc style centrifuge 2 (step 748) and the overflow from protein decanter 2 (step 747) to the disc style centrifuge 1 (step 746) or used as backset to save energy. The protein cake from protein decanter 1 (step 735) and protein decanter 2 (step 747) is sent to a protein dryer (step 771) to produce a high protein meal with up to 50% protein content and a yield of up to 6 lb./Bu protein meal. The fiber cake from the fiber dewater process (step 44) is directed to the DDG dryer (step 772). The enriched syrup can be added to the DDG dryer before, during, or after the dryer has finished drying the DDG. The addition of enriched syrup produces enriched DDGS with high organic acid concentration, such as lactic acid, along with probiotic character high protein meal. In some embodiments, a small portion of the enriched syrup is added to the protein meal to produce an enriched protein meal with high organic acid, such as lactic acid and probiotic character.

In some embodiments, the process 700A of FIG. 7A is adding the whole stillage feed directly to particle size separation (step 741A) and rest of the process steps in process 700A is same as process 700 in FIG. 7 in some embodiments. Two disc centrifuge conjunction with two decanter centrifuge will produce cleanest thin stillage with less than 0.5% solid by volume and produce 80% DS or more syrup.

The process and system disclosed herein can start from using whole stillage, which is produced at the distillation step (e.g., separated by a fiber separation process, such as the step 15 of FIG. 1). In some embodiments, the whole stillage includes the insoluble solids (“wet cake”.) The liquid from the distillation step (e.g., separated by a fiber separation process, such as the step 15 of FIG. 1) is referred to as thin stillage.

The backend grinding system as shown in FIG. 4, is disclosed in the patent (U.S. Pat. No. 9,388,475, is titled “SYSTEM FOR AND METHOD OF SEPARATING OIL AND PROTEIN FROM GRAINS USED FOR ALCOHOL PRODUCTION” which is incorporated by reference in its entirety for all purposes).

To be succinct, not all the process and steps are repeated in the descriptions. For example, each and every step described in FIGS. 1 to 4 are able to be entirely or selectively applied to any of the figures herein. Each of the processes and steps disclosed herein can be selected to be performed or omitted.

In utilization, the process is used to make a high syrup concentration using a dry mill process.

In operation, the whole stillage from the distiller is going through the dry mill process with advanced features including a) a backend grinding to increase alcohol, oil, and protein yield, b) two-disc centrifuges in series to produce cleanest thin stillage, c) a secondary fermentation to produce enriched syrup with the highest % DS syrup, d) splitting two dryers in series to two dryers in parallel to produce high protein meal and enriching low protein feed to improve the syrup concentration.

While the present invention has been illustrated by a description of various embodiments and while these embodiments have been described in considerable detail, it is not the intention of the applicant to restrict or in any way limit the scope of the appended claims to such detail. For example, although the various systems and methods described herein have focused on corn, virtually any type of grain, including, but not limited to, wheat, barley, sorghum, rye, rice, oats and the like, can be used. The purified fiber, often called white fiber, can be used for a number of applications including for the paper industry or as feed stock for secondary (“cellulosic”) alcohol production. Thus, the invention in its broader aspects is therefore not limited to the specific details, representative apparatus and method, and illustrative example shown and described. Accordingly, departures may be made from such details without departing from the spirit or scope of applicant's general inventive concept.

The present invention has been described in terms of specific embodiments incorporating details to facilitate the understanding of principles of construction and operation of the invention. Such reference herein to specific embodiments and details thereof is not intended to limit the scope of the claims appended hereto. For example, the disc centrifuge can be a nozzle centrifuge, a desludging centrifuge, disc decanter, sedicanter or other suitable high g centrifuge device as may be available today or in the future. The particle size reduce device can be disk grinding mill, roller mill, collider mill, pin mill or any other type of suitable milling equipment. It will be readily apparent to one skilled in the art that other various modifications may be made in the embodiment chosen for illustration without departing from the spirit and scope of the invention as defined by the claims.

What is claimed is:

1. A method of generating concentrated syrup in a dry milling system comprising:
a. fermenting and distilling an agricultural substance;
b. forming a whole stillage;
c. performing a first separating process on the whole stillage;
d. forming an overflow liquid based portion and underflow coarse solid portion at the first separating process;
e. grinding the underflow coarse solid portion after the first separating process;
f. using one or more high speed centrifuges for performing a centrifugation process on the overflow liquid based portion;
g. evaporating the overflow liquid based portion at an evaporator from the one or more high speed centrifuge; and
h. forming a concentrated syrup.

2. The method of claim 1, generating a solution at the having less than 2% of insoluble solid by using a high G force nozzle centrifuge at the centrifugation process.

3. The method of claim 2, wherein the high G force nozzle centrifuge with the evaporator are able to concentrate the overflow liquid based portion to a syrup having 50%-60% of dry matter.

4. The method of claim 1, wherein the one or more high speed centrifuges comprises a nozzle disc stack centrifuge, which generates a solution having 0.6%-1.2% of insoluble solid.

5. The method of claim 4, wherein the nozzle disc stack centrifuge with the evaporator are able to concentrate the overflow liquid based portion to a syrup having 60%-70% of dry matter.

6. The method of claim 1, wherein the one or more high speed centrifuges comprises double high-speed centrifuges.

7. The method of claim 6, wherein the double high-speed centrifuges comprise a high-speed disc centrifuge and a high-speed disc stack centrifuge.

8. The method of claim 7, wherein the high-speed disc stack centrifuge generates a solution having 0.25%-4% of insoluble solid by volume.

9. The method of claim 6, wherein the double high-speed centrifuges with the evaporator are able to concentrate the overflow liquid based portion to a syrup having greater than 70% of dry matter.

10. The method of claim 6, wherein the double high-speed centrifuges with the evaporator are able to concentrate the overflow liquid based portion to a syrup having 70%-80% of dry matter.

11. The method of claim 1, wherein the one or more high speed centrifuges comprises a disc decanter.

12. The method of claim 11, wherein the a disc decanter with the evaporator are able to concentrate the overflow liquid based portion to a syrup having 70%-80% of dry matter.

13. The method of claim 11, further comprising reducing a viscosity of the syrup by converting glycerol and residual sugars in the syrup to one or more organic acids.

14. A method of generating concentrated syrup in a dry milling system comprising:
a. fermenting and distilling an agricultural substance;
b. forming a whole stillage;
c. performing a first separating process on the whole stillage;
d. forming an overflow liquid based portion and underflow coarse solid portion at the first separating process;
e. grinding the underflow coarse solid portion after the first separating process;
f. using one or more high speed centrifuges for performing a centrifugation process on the overflow liquid based portion;
g. evaporating the overflow liquid based portion at an evaporator from the one or more high speed centrifuge
h. forming a concentrated syrup; and
adding the concentrated syrup back to the evaporator to form a syrup having 80% to 90% of a dry matter.

15. The method of claim 1, wherein the one or more high speed centrifuges comprises two disc centrifuges.

16. The method of claim 14, wherein the dry milling system does not use a drying temperature that destroys heat sensitive nutrients in the syrup.

17. The method of claim 14, further comprising using two parallel dryer lines.

18. The method of claim 17, wherein the two parallel dryer lines produce two different protein contents.

19. A method of increasing a percentage of dry matter in a syrup in a dry milling alcohol producing plant comprising:
a. reducing a viscosity of a syrup by reducing a percentage of the insoluble solid in a thin tillage; and
b. evaporating water forming a syrup with a dry matter greater than 50% in an evaporator without causing a significant flow and wetting issue that form an amount of scale at an evaporator tube of the evaporator.

20. The method of claim 20, wherein the insoluble solid is less than 2% in the thin stillage.

21. The method of claim 20, further comprising using one or more centrifuges to reduce the percentage of the insoluble solid.

22. The method of claim 22, wherein the one or more centrifuges comprises a high G force nozzle centrifuge.

23. The method of claim 22, wherein the one or more centrifuges comprises a nozzle disc stack centrifuge.

24. The method of claim 22, wherein the one or more centrifuges comprises double high-speed centrifuges.

25. The method of claim 22, wherein the one or more centrifuges comprises a disc decanter.
